(19) United States
(12) Patent Application Publication
(10) Pub. No.: US 2012/0101579 A1
(43) Pub. Date: Apr. 26, 2012

(54) PROSTHETIC INTERVERTEBRAL DISC WITH MOVABLE CORE

(75) Inventors: Malan de Villiers, Wapadrand (ZA); Neville Jansen, Waterkloof (ZA)

(73) Assignee: SpinalMotion, Inc., Mountain View, CA (US)

(21) Appl. No.: 13/094,131

(22) Filed: Apr. 26, 2011

Related U.S. Application Data
(60) Provisional application No. 61/328,504, filed on Apr. 27, 2010.

(51) Int. Cl. A61F 2/44

(52) U.S. Cl. 623/17.16

(57) ABSTRACT

An intervertebral disc for a prosthetic intervertebral disc system for insertion from a posterior of the spine includes first and second end plates sized and shaped to fit within an intervertebral space, each end plate having a vertebral contacting surface and an inner surface, a first bearing surface on an inner surface of the first end plate, and a second bearing surface on an inner surface of the second end plate. A mobile core is configured to be received between the first and second bearing surfaces, the first and second end plates being articulable and rotatable relative to each other via sliding motion of at least one of the first and second bearing surfaces over the core. The core is movable with respect to at least one of the first and second end plates.

### CROSS-REFERENCE TO RELATED APPLICATION

This application claims the benefit of U.S. Provisional Application No. 61/328,504 (Attorney Docket No. 022031-005300US), filed Apr. 27, 2010, the full disclosure of which is incorporated herein by reference.

### BACKGROUND AND SUMMARY OF THE INVENTION

The present invention relates to medical devices and methods. More specifically, the invention relates to intervertebral prosthetic discs and methods of preserving limited motion upon removal of an intervertebral disc.

Back pain takes an enormous toll on the health and productivity of people around the world. According to the American Academy of Orthopedic Surgeons, approximately 80 percent of Americans will experience back pain at some time in their life. In the year 2000, approximately 26 million visits were made to physicians' offices due to back problems in the United States. On any one day, it is estimated that 5% of the working population in America is disabled by back pain.

One common cause of back pain is injury, degeneration and/or dysfunction of one or more intervertebral discs. Intervertebral discs are the soft tissue structures located between each of the thirty-three vertebral bones that make up the vertebral (spinal) column. Essentially, the discs allow the vertebrae to move relative to one another. The vertebral column and discs are vital anatomical structures, in that they form a central axis that supports the head and torso, allow for movement of the back, and protect the spinal cord, which passes through the vertebrae in proximity to the discs.

Discs often become damaged due to wear and tear or acute injury. For example, discs may bulge (herniate), tear, rupture, degenerate or the like. A bulging disc may press against the spinal cord or a nerve exiting the spinal cord, causing “radicular” pain (pain in one or more extremities caused by impingement of a nerve root). Degeneration or other damage to a disc may cause a loss of “disc height,” meaning that the natural space between two vertebrae decreases. Decreased disc height may cause a disc to bulge, facet loads to increase, two vertebrae to rub together in an unnatural way and/or increased pressure on certain parts of the vertebrae and/or nerve roots, thus causing pain. In general, chronic and acute damage to intervertebral discs is a common source of back related pain and loss of mobility.

When one or more damaged intervertebral disc cause a patient pain and discomfort, surgery is often required. Traditionally, surgical procedures for treating intervertebral discs have involved discectomy (partial or total removal of a disc), with or without interbody fusion of the two vertebrae adjacent to the disc. When the disc is partially or completely removed, it is necessary to replace the excised disc material with natural bone or artificial support structures to prevent direct contact between hard bony surfaces of adjacent vertebrae. Oftentimes, pins, rods, screws, cages and/or the like are inserted between the vertebrae to act as support structures to hold the vertebrae and any graft material in place while the bones permanently fuse together.

A more recent alternative to traditional fusion is total disc replacement or TDR. TDR provides the ability to treat disc related pain without fusion provided by bridging bone, by using a movable, implantable, artificial intervertebral disc (or “disc prosthesis”) between two vertebrae. A number of different artificial intervertebral discs are currently being developed. For example, U.S. Pat. Nos. 7,442,211 and 7,531,001 and U.S. Patent Application Publication Nos. 2005/0021145, 2007/0282449 and 2010/0016973 which are hereby incorporated by reference in their entirety, describe artificial intervertebral discs with mobile bearing designs. Other examples of intervertebral disc prostheses are the Charité® disc (provided by DePuy Spine, Inc.) MOBIDISC® (provided by LDR Medical (www.ldrmedical.fr)), the BRYAN Cervical Disc (provided by Medtronic Sofamor Danek, Inc.), the PRODISC® or PRODISC-C® (from Synthes Stratec, Inc.), the PCM disc (provided by Cervitech, Inc.), and the MAVERICK® disc (provided by Medtronic Sofomor Danek).

A potential drawback of many of these known disc designs is that the prosthetic disc must be inserted from the anterior side of the patient. The anterior approach can be difficult and may require a vascular surgeon as the prosthetic disc passes near important blood vessels located anterior to the spine. Other currently available intervertebral disc prostheses usually have similar drawbacks, including invasiveness of the surgery and/or surgical skill and complexity.

Another prosthetic approach has been to fuse the vertebrae, for example with transforaminal lumbar interbody fusion (TLIF) surgery or posterior lumbar interbody fusion (PLIF) surgery. These procedures allow the surgery to be performed from the posterior without passing through the abdominal cavity and the associated drawbacks. The TLIF or PLIF approaches involve passing through a much smaller space than an anterior approach and generally require at least partial removal of one or more facet joints to provide enough space for access to the disc space. It is this limitation on space that has until now prevented the design of a successful artificial disc for delivery by a TLIF or PLIF approach.

Therefore, a need exists for an improved disc for preserving motion and maintaining disc spacing between two vertebrae after removal of an intervertebral disc which can be delivered by a TLIF or PLIF approach. Ideally, such improved disc would be introduced in a small configuration.

In accordance with one aspect of the invention, an intervertebral disc for a prosthetic intervertebral disc system for insertion from a posterior of the spine is provided. The disc comprises first and second end plates sized and shaped to fit within an intervertebral space, each end plate having a vertebral contacting surface and an inner surface, a first bearing surface on an inner surface of the first end plate, a second bearing surface on an inner surface of the second end plate, and a mobile core configured to be received between the first and second bearing surfaces, the first and second end plates being articulable and rotatable relative to each other via sliding motion of at least one of the first and second bearing surfaces over the core, and the core being movable with respect to at least one of the first and second end plates.

In accordance with another embodiment of the invention, a prosthetic intervertebral disc system for insertion from a posterior of the spine is provided. The disc system comprises first and second intervertebral discs configured to be inserted from a posterior of the spine, each of the first and second intervertebral discs comprising first and second end plates sized and shaped to fit within an intervertebral space, each end plate having a vertebral contacting surface and an inner surface, a first bearing surface on an inner surface of the first end plate, a second bearing surface on an inner surface of the second end plate, and a mobile core configured to be received between the first and second bearing surfaces, the first and second end plates being articulable and rotatable relative to each other via sliding motion of at least one of the first and second bearing surfaces over the core, and the core being movable with respect to at least one of the first and second end plates.

In accordance with a further aspect of the invention, a prosthetic intervertebral disc system for insertion between two vertebrae from a posterior of the spine is provided. The disc system comprises a first intervertebral disc including a first end plate having a length no greater than a length from the anterior to the posterior of the vertebrae, a second end plate coordinating with the first end plate and a first rigid core configured to be movably seated between the first and second end plates, and a second intervertebral disc including a third end plate having a length no greater than a length from the anterior to the posterior of the vertebrae, a fourth end plate coordinating with the third end plate and a second rigid core configured to be movably seated between the third and fourth end plates.

In accordance with a further aspect of the invention, a method of inserting a prosthetic intervertebral disc from a posterior of the spine is provided. The method comprises accessing an intervertebral disc space between a first and second vertebrae from a posterior of a patient, and inserting first and second intervertebral discs from the posterior of the patient on first and second sides of a midline of the spine, wherein the first and second intervertebral discs each have a mobile core.

### BRIEF DESCRIPTION OF THE DRAWINGS

The features and advantages of the present invention are well understood by reading the following detailed description in conjunction with the drawings in which like numerals indicate similar elements and in which:

FIG. 1 is a perspective view of a spine with a prosthetic intervertebral disc system implanted therein according to an aspect of the present invention;

FIG. 2 is a perspective view of the prosthetic intervertebral disc system of FIG. 1;

FIG. 3 is a perspective view of a prosthetic intervertebral disc system according to an aspect of the present invention;

FIG. 4 is a perspective view of a portion of a tool for insertion of a prosthetic intervertebral disc according to an aspect of the present invention;

FIG. 5 is a perspective view of a portion of a tool for insertion of a prosthetic intervertebral disc according to an aspect of the present invention;

FIG. 6 is a cross-sectional view of a prosthetic intervertebral disc taken at section 6-6 of FIG. 2;

FIG. 7A is a top view of a prosthetic intervertebral disc with end plates rotated relative to each other;

FIG. 7B is a side view of a prosthetic intervertebral disc with end plates moved 12° in the extension direction;

FIG. 7C is a side view of a prosthetic intervertebral disc with end plates moved 12° in the flexion direction; and

FIG. 8 is an exploded view of a prosthetic intervertebral disc and portions of a tool for insertion of the disc according to an aspect of the present invention.

### DETAILED DESCRIPTION

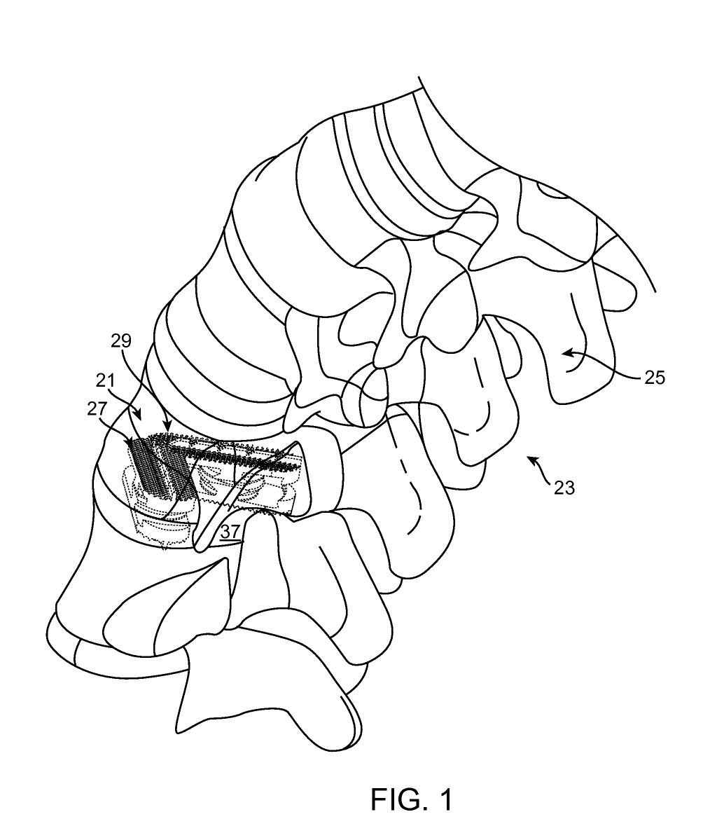

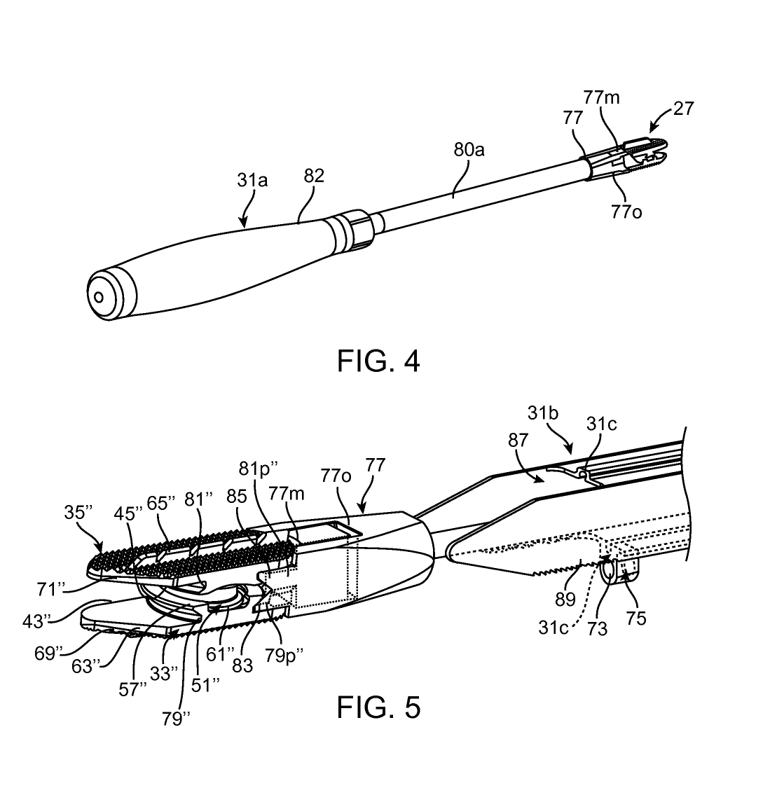

A prosthetic intervertebral disc system 21 for insertion from a posterior 23 of the spine 25 is shown in FIG. 1 The disc system 21 comprises first and second intervertebral discs 27 and 29 that are each configured to be inserted from the posterior 23 of the spine 25 independently from two insertion locations oriented at an angle to one another. The two discs 27 and 29 can be sized to avoid or minimize removal of facets on the spine during insertion of the discs, such as by requiring only removal of a small slice at a medial side of each facet. To this end, the discs 27 and 29 can have substantially elongated, narrow shapes such as in the aspects of the system seen in FIGS. 1-3, and can be inserted using thin tools such as an insertion instrument 31a and guide 31b, such as are shown in FIGS. 4 and 5, respectively.

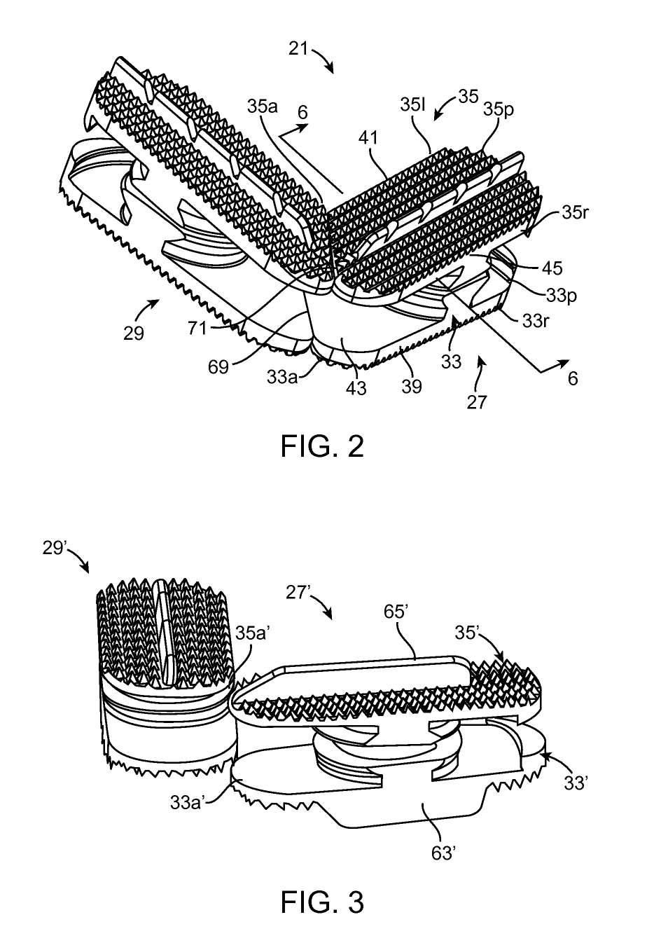

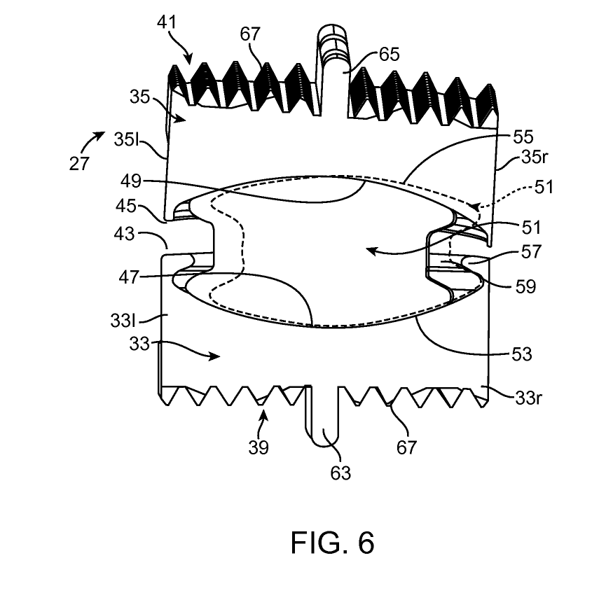

The first and second discs 27 and 29 can be mirror images of each other, as seen in FIGS. 1 and 2, or the first and second discs 27′ and 29′ can be identical, as seen in FIG. 3. FIG. 6 shows a cross-section of the disc 27 for purposes of illustration, it being understood that all of the discs 27, 29 (FIGS. 1 and 2), 27′, 29′ (FIG. 3), and 27″ (FIG. 5) can have an identical or substantially similar cross-section, except where otherwise noted.

The disc 27 comprises first and second end plates 33 and 35 that are sized and shaped to fit within an intervertebral space 37 (FIG. 1). As seen, for example, in FIG. 2, each end plate 33 and 35 has a vertebral contacting surface 39 and 41, respectively, and an inner surface 43 and 45, respectively. As seen in FIG. 6, the first end plate 33 has a first bearing surface 47 on the inner surface 43 of the first end plate. The second end plate 35 has a second bearing surface 49 on the inner surface 45 of the second end plate.

A mobile core 51 is configured to be received between the first and second bearing surfaces 47 and 49. The core 51 has at least one, ordinarily two, curved bearing surfaces 53 and 55. The core 51 is ordinarily rigid, i.e., substantially inflexible and incompressible, however, the core may be flexible, compressible, and/or compliant with rigid bearing surfaces. In the embodiment shown in FIG. 6, the core 51 is rigid and has first and second curved bearing surfaces 53 and 55 for contacting the first and second curved bearing surfaces 47 and 49, respectively, of the first and second end plates. The first and second end plates 33 and 35 are articulable and rotatable relative to each other via sliding motion of at least one of, ordinarily both of, the first and second bearing surfaces 47 and 49 over the core 51. In one embodiment of an intervertebral disc system designed for use in the lumbar spine, a core has a diameter of about 10-15 mm, a height of about 5-10 mm, and a spherical radius of curvature of the bearing surfaces of about 10-18 mm, in one preferred embodiment the core has a diameter of about 12.5 mm, a height of about 7.2 mm, and a spherical radius of about 14 mm. The intervertebral discs 27 and 29 may be provided in different heights to accommodate patient anatomy. For example, discs may be provided in multiple heights, such as of 9, 11 and 13 mm or in other size variations. The discs may also be provided in different sizes. In one example, for example different lengths in the anterior/posterior direction can be used to accommodate different anatomies. Although different width discs can be used, in the preferred embodiment, the width of the discs is approximately the same as the width of the mobile core, i.e. about 10-15 mm, preferably about 12.5 mm. The same core 51 and bearing surface configuration is preferably use in the discs of multiple sizes.

As shown in FIG. 2, the first and second end plates 33 and 35 each have left and right longitudinal sides 33l and 33r and 35l and 35r, when the disc is viewed from the anterior, and anterior and posterior ends 33a and 33p and 35a and 35p. The anterior ends 33a and 35a may be angled or formed in a complementary shape which allows the anterior ends to mate with each other upon insertion. Alternately, the anterior ends, as shown in FIG. 3, may be configured in such a shape that they touch without mating or do not touch in the implanted configuration.

At least the first end plate 33 includes a projection 57 which engages with a recess 59 of the core 51 to retain the core between the first and second end plates 33 and 35. The projection 57 can be in the form of an annular rim and the recess 59 can be in the form of an annular groove. In the embodiments shown in FIGS. 1-3 and 6, the projection 57 extends 360° around the core 51. The inner diameter DP of the projection 57 is ordinarily equal to or greater than a greatest diameter DC of the core 51 so that, during assembly, part of the core below the recess 59 can be passed through an opening defined by the projection. In the embodiment shown in FIG. 5, the projection 57″ only extends part of the way around the core 51″, which can facilitate providing a larger diameter core in a narrower disc 27″. In all embodiments, however, the core and the projection are ordinarily shaped so that the part of the core below the recess can be passed through an opening defined by the projection in a direction substantially along an axis of the opening defined by the projection. For example, in the disc 27″ shown in FIG. 5, the core cannot ordinarily be removed through the side opening 61″. In other words, the side opening 61″ of the projection has a length which is less than the diameter of the core. In the embodiments illustrated, the first end plate 33 having the retaining ring or projection 57″ has been illustrated as the inferior end plate. However, it should be understood that the first end plate may be positioned as the inferior or superior end plate.

The second end plate can be provided with a projection instead of or in addition to having the projection on the first end plate. Ordinarily, however, the second end plate will not have a projection, which can reduce the thickness of the disc and increase range of motion of the second end plate relative to the core and the end plates relative to each other.

In addition to permitting sliding motion, the core 51 is movable with respect to both of the first and second end plates 33 and 35. “Movable” is specifically defined for purposes of describing the movability of the core 51 with respect to at least one of the first and second end plates 33 and 35 as meaning that the core is adapted to be displaced in a direction toward or away from at least one of the left and right longitudinal sides 33l and 33r and 35l and 35r and anterior and posterior ends 33a and 33p and 35a and 35p of at least one of first and second end plates. The core 51 is movable with respect to both the first and second end plates when the intervertebral disc system is in use implanted between the vertebrae of a patient. However, the core 51 may have a non-movable position when the disc is being implanted or before completion of a surgical implantation procedure.

FIG. 6 shows, in phantom, the core 51 moved toward the right longitudinal sides 33r and 35r of both the first and second end plates 33 and 35 relative to the position of the core 51 shown in solid lines. While not shown, the angular position of the first and second end plates 33 and 35 would likely be different from that shown if the core 51 moved to the position shown in phantom. Although the radius of curvature of the bearing surfaces of the core and the bearing surfaces of the plates 47 and 49, as shown, are substantially the same (substantially congruent) for purposes of distribution of load and reduced wear, in other embodiments, at least one of the first and second bearing surfaces 47 and 49, or the bearing surfaces of the core can be modified with a groove, channel, depression or flat on a portion of the bearing surface.

Instead of providing two curved bearing surfaces on the core and corresponding curved bearing surfaces on the first and second end plates, one of the bearing surfaces may be another shape, such as substantially flat (not shown). For example, the first bearing surface on the core below the recess can be flat and the first bearing surface on the first end plate below the projection can be flat. The first bearing surface on the first end plate can be larger than the first bearing surface on the core to facilitate limited translational movement of the core relative to the first end plate. If the second bearing surface on the second end plate and the second bearing surface on the core are curved, the first and second end plates will be articulable and rotatable relative to each other via sliding motion. In another example, the first bearing surface 47 on the inner surface 43 of the first end plate and corresponding bearing surface 53 on the core 51 can be flat while the second bearing surface 49 on the second inner surface 45 of the second end plate and the corresponding bearing surface 55 of the core can be cylindrically curved in a direction to allow anterior posterior rotation of the upper endplate. Other bearing surface shapes which can also be used depending on the type of motion of the mobile core desired including trough shaped, kidney bean shaped, elliptical, or oval bearing surfaces.

As seen, for example, in FIG. 6, the first and second end plates 33 and 35 can each include one or more elongated fin or keel 63 and 65, respectively, on the vertebral contacting surface 39 and 41, respectively, thereof. If a single, central fin is provided, there will be less cutting of bone required during insertion. However, if multiple, usually two, fins are provided on one or more of the end plates, there can be more bone contact between the fins and increased fixation. Often, two fins are provided on a bottom end plate and one on top to improve fixation of the lower plate with respect to the bone, as the lower plate has been found to be the plate with a greater tendency to move relative to the spine. Also, providing fins that are not aligned, such as by providing, as shown in FIG. 3, one top, central fin 65′ on the second end plate 35′ and one or two bottom fins 63′ displaced to an outside of the first end plate 33′, can facilitate performing two level disc replacements for discs on opposite sides of the same vertebra because the cuts that must be made for the fins are staggered. The different fin arrangements on the first and second end plates can also assist in determining a proper orientation for the disc. The vertebral contacting surfaces 39 and 41 may, in addition to the keels, be provided with knurling, teeth, serrations 67 or some other textured surface to increase friction between the vertebral contacting surfaces and the adjacent vertebra. The fins 63, 63′ and 65 as shown have an angled anterior end for ease of insertion and a plurality of angled slots for improved bone fixation.

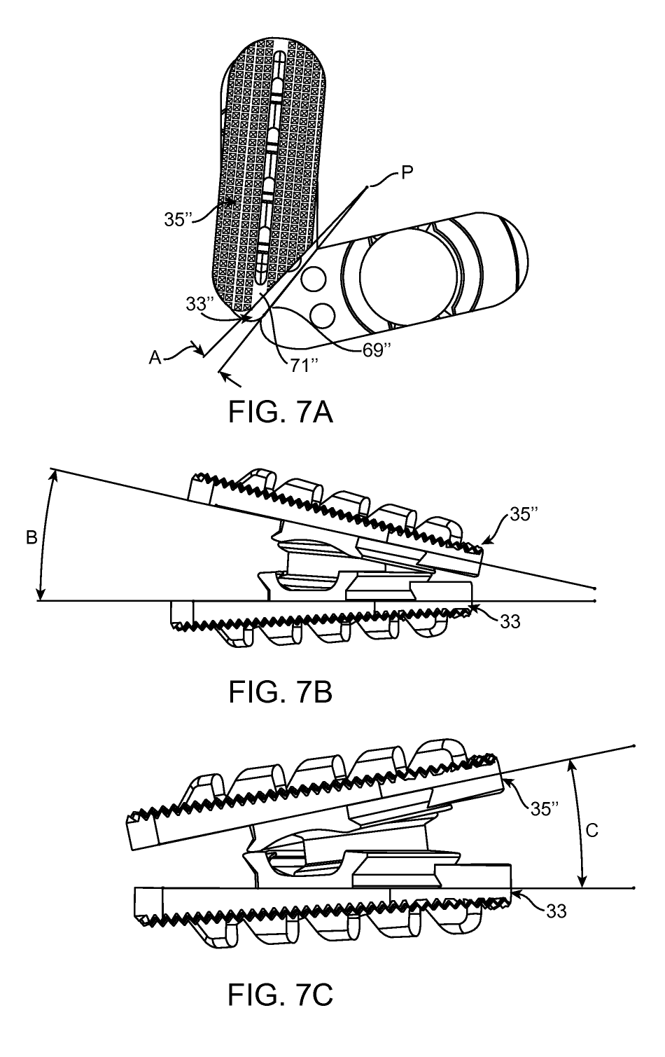

FIG. 7A shows end plate 35″ rotated relative to end plate 33″ so that the flat surfaces 71″ and 69″ on the anterior ends of the end plates form an angle A measured from the central axis P located approximately at the center of the vertebral surface. The top end plate of the neighboring disc has been removed in FIG. 7A for purposes of illustration. In one example, the first and second end plates and can be configured to be arranged within a disc space to provide axial rotation of about ±2 to ±12 degrees, and preferably about ±5 degrees. This ability of the intervertebral disc system to allow some rotation between adjacent vertebrae provides an improvement over a ball and socket device provided with two ball and socket articulations which does not allow rotation. The amount of rotational motion can be increased by increasing a range of motion of each of the cores.

The first and second end plates 33 and 35 can be configured to be arranged within a disc space to provide motion in the flexion/extension direction up to an angle B. In one example, the angle B is about ±5 to ±15 degrees, and preferably about ±12 degrees of motion in the flexion/extension direction, i.e., relative angular movement of the anterior and posterior ends 33a and 33p (FIG. 1) of the first end plate with respect to the anterior and posterior ends 35a and 35p (FIG. 1) of the second end plate. FIG. 7B shows the end plates 33″ and 35″ moved 12° in the extension direction. 7C shows the end plates at an angle C or moved 12° in the flexion direction. In a presently preferred disc system, for implants placed with the center of the concave bearing surfaces 47, 49 of the endplates of the two discs 27 and 29 of the disc system about 22 mm apart, the following kinematics can be expected: ±5 degrees of axial rotation and ±12 degrees of flexion/extension. The intervertebral disc system self centers due to the fact that as the core moves away from a neutral centered position, the assembled height of the system gradually increases. The force of the surrounding tissue try to bring the disc back to the lower height configuration of the neutral position. One or both of the end plates can be provided in a selection of lordotic angles with the end plates having a wedge shape (see FIGS. 7B and 7C) to allow a surgeon to select a desired lordosis for a particular patient anatomy. In one example, discs can be provided in lordotic angles of 0°, 5° and 10°.

FIGS. 1 and 2 show first and second intervertebral discs 27 and 29 that are mirror images of each other. The end plates 33 and 35 of the disc 27 have flattened diagonal surfaces 69 and 71 (see also 69″ and 71″ in FIG. 5) at their anterior ends 33a and 35a that are intended to abut with flattened diagonal surfaces at the anterior ends of the disc 29 when the discs are implanted in an intervertebral space, which can permit more coverage on the endplates, i.e., less chance of subsidence of the disc into the bone.

FIG. 3 shows an alternative arrangement wherein the discs 27′ and 29′ are identical and the end plates 33′ and 35′ are intended to be spaced apart or only contact at one point when the discs are inserted in an intervertebral space. In FIG. 3, the anterior ends 33a′ and 35a′ of the end plates 33′ and 35′ are substantially rounded so that contact between the anterior ends of the end plates of the discs 27′ and 29′ can occur, if at all, only at one point. This may be desirable if it is desired to vary the implant angle of the discs 27′ and 29′ with respect to one another or to allow different insertion angles to accommodate differing patient anatomies. The arrangement of mating angled anterior ends of FIGS. 1 and 2 can provide some additional vertebral body endplate coverage than the disc of FIG. 3 with non angled anterior ends.

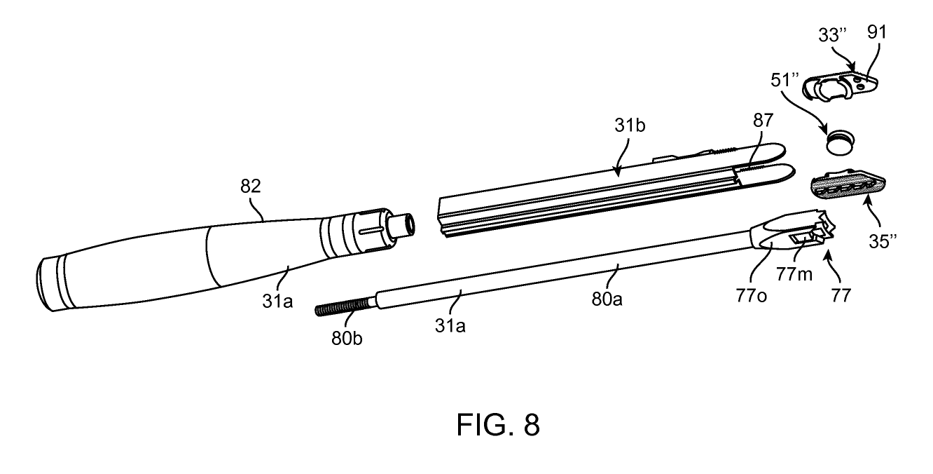

FIGS. 4 and 5 show the insertion instrument 31a for use in a method of inserting a prosthetic intervertebral disc from a posterior 23 (FIG. 1) of the spine 25 (FIG. 1). FIG. 5 also shows a portion of a guide 31b for use with the insertion instrument 31a. FIG. 8 shows components of the insertion instrument 31a and guide 31b in an exploded configuration. Broadly, a method of inserting the intervertebral disc 21 comprises accessing an intervertebral disc space between a first and second vertebra from a posterior of a patient. First and second intervertebral discs having a mobile core (e.g., 27 and 29 in FIG. 1) are inserted from the posterior of the patient on first and second sides of a midline of the spine. By this method, motion can be provided between the first and second vertebrae in the directions of flexion/extension and axial rotation. The first and second intervertebral discs can be inserted and placed in the intervertebral disc space without contacting one another, such as when using discs 27′ and 29′ as shown in FIG. 3 so that the first and second intervertebral discs are not connected to one another in the intervertebral disc space. Alternatively, as seen in FIG. 2, the first and second discs 27 and 29 can be inserted and placed in the disc space so that surfaces 69 and 71 at anterior ends 33a and 35a of the first and second end plates 33 and 35 of the first disc abut mirror image surfaces at anterior ends of the first end plate and the second end plate of the second disc.

To implant a disc (e.g., the disc 27″ shown in FIG. 5), a bi-lateral Wiltse approach can be used to access the posterior of the spine. The procedure for replacing a natural intervertebral disc with the artificial intervertebral discs includes using a PLIF or TLIF approach to the spine, by forming a 3-6 inch incision in the patient's back and retracting the spinal muscles to allow access to the vertebral disc. The surgeon then carefully removes the lamina (laminectomy) to be able to see and access the nerve roots. The disc space is then entered through a preexisting opening or through an opening formed by cutting away a small portion of one or more facets. Depending on the amount of space available some portion of the facets may be removed, for example 0% to 50% of the facets may be removed, however preferably less than 30% of the facets are removed. Those skilled in the art will understand the procedure of preparing the disc space and implanting the disc which is summarized herein. A far posterio-lateral minimally invasive approach can be used so as to allow for the minimum of facet removal such that the facet joints remain substantially intact.

After removal of the natural disc material, sequentially larger dilators (not shown) are used to gradually increase disc height of the intervertebral space. A sizing tool (not shown) having an end (referred to here as a “trial”) that can be the shape and size of the disc 27″ can be positioned in the intervertebral space to determine a size disc to be implanted. A trial will ordinarily be positioned in the intended location for both of the discs of the disc system either independently or together and viewed under X-ray. With the trials in place, the guide 31b is placed over the trials and positioned in place for insertion of the disc. The guide 31b can then be locked in place with a bone screw (not shown) that is passed through a screw hole 73 on a protrusion 75 extending radially from the guide (FIG. 5). The guides 31b fixed at both sides of the posterior of the spine will provide a tunnels through which two discs are inserted.

FIG. 5 shows components of the guide 31b and the insertion instrument 31a as transparent components for purposes of illustration of the features of the guide and handle. After the guide 31b is secured in a desired position, the trials can then be removed through the channel 87 of respective guides 31b by their handles. Fin or keel cutters (not shown) can be passed through the guides 31b to form recesses for receiving the fins 63″ and 65″. The guides 31b are provided with upper and lower channels 31c for receiving the keel cutters and subsequently, the keels 65 of the discs. The number, shape and size of the upper and lower channels 31c correspond to the keels on the discs. The guides 31b stay in place throughout the disc implantation procedure to protect the nerve roots which exit the spinal cord near the working area and to protect other surrounding structures. The guides 31b may be formed of a signal conducting material and may be connectable to appropriate nerve monitoring equipment, such as NeuroVision®, the NuVasive nerve avoidance monitoring system.

The insertion instrument 31a shown in FIGS. 4 and 8 is adapted to hold a disc (e.g., disc 27 shown in FIG. 4) proximate a posterior end of the disc at a forward end 77 of the insertion instrument, part of which is shown in FIG. 5. As seen with respect to the disc 27″ shown in FIG. 5, grooves 79″ and 81″ are provided on the inner surfaces 43″ and 45″, respectively, of the first end plate 33″ and second end plate 35″, respectively. As seen in FIGS. 5 and 8, the forward end 77 of the insertion instrument includes a movable middle portion 77m with a downwardly extending hook portion 83 (FIG. 5) that is received in the groove 79″, and that is pulled back toward the handle to hold the lower (first) end plate on the insertion instrument. To release the disc, the hook portion 83 is moved forward away from the handle of the insertion instrument and the hook is unhooked from the lower end plate. A fixed outer portion 77o extends along the outside surfaces of the disc to further retain the disc on the insertion instrument. The upper (second) end plate is not held in place by the hook portion 83 and rests between the fixed outer portion 77o and when the disc is within the guide is retained by the guide and guided by the channel 31c. After the disc is in position in the intervertebral space, an interior wedge shaped portion of the insertion instrument 31a outer portion 77o biases against the posterior edge of the disc and distract the end plates sufficiently to allow removal of the hook portion 83 from the disc groove.

As seen in FIG. 8, the insertion instrument 31a can include an outer part 80a attached to the outer portion 77o of the forward end 77 of the handle and an inner part 80b attached to the middle portion 77m. The outer and inner parts 80a and 80b can be axially movable relative to each other so that movement of the inner part 80b relative to the outer part 80a moves the middle portion 77m of the forward end 77 of the handle 31a relative to the outer portion 77o. The insertion instrument 31a may be provided with a grip 82 (FIG. 8) that is axially fixed but rotatable relative to the outer part 80a and that includes an internally threaded portion that mates with an externally threaded portion of the inner part 80b so that turning of the grip 82 relative to the outer part moves the inner part 80b and, thus, the attached middle portion 77m relative to the outer part 80a and the attached outer portion 77o. Other systems for moving the middle portion relative to the outer portion of the insertion instrument may also be used.

The protrusions 83 and 85 hold the disc 27″ by the grooves and facilitate pushing the disc with the end plates 33″ and 35″ spaced at least a minimum desired amount through a channel 87 of the guide 31b and into an implantation position in the intervertebral space. The channel 87 ensures that the end plates remain in a parallel position relative to each other during implantation. The guide 31b can have teeth 89 at a forward end to facilitate gripping of the vertebra. The posterior walls of the grooves 79″ and 81″ can be sloped to facilitate secure grasping with the hook shaped portion of the insertion instrument. After the discs are placed, the insertion instruments 31a and the guides 31b are removed from the patient.

As seen in FIG. 8, the end plates can be provided with holes or recesses 91 at an anterior end of the end plates to facilitate removal of the end plates anteriorily in a subsequent surgical procedure if needed.

The first and second end plates of the two discs may be constructed from any suitable metal, alloy or combination of metals or alloys, such as but not limited to cobalt chrome alloys, titanium (such as grade 5 titanium), titanium based alloys, tantalum, nickel titanium alloys, stainless steel, and/or the like. They may also be formed of ceramics, biologically compatible polymers including PEEK, UHMWPE, PLA or fiber reinforced polymers. The end plates may be formed of a one piece construction or may be formed of more than one piece, such as different materials coupled together.

The core can be made of low friction materials, such as titanium, titanium nitrides, other titanium based alloys, tantalum, nickel titanium alloys, stainless steel, cobalt chrome alloys, ceramics, or biologically compatible polymer materials including PEEK, UHMWPE, PLA or fiber reinforced polymers. High friction coating materials can also be used.

Different materials may be used for different parts of the disc to optimize imaging characteristics. PEEK end plates may also be coated with titanium plasma spray or provided with titanium screens for improved bone integration. Other materials and coatings can also be used such as titanium coated with titanium nitride, aluminum oxide blasting, HA (hydroxylapatite) coating, micro HA coating, and/or bone integration promoting coatings. Any other suitable metals or combinations of metals may be used as well as ceramic or polymer materials, and combinations thereof. Any suitable technique may be used to couple materials together, such as snap fitting, slip fitting, lamination, interference fitting, use of adhesives, welding and/or the like.

In the present application, the use of terms such as “including” is open-ended and is intended to have the same meaning as terms such as “comprising” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

While this invention has been illustrated and described in accordance with a preferred embodiment, it is recognized that variations and changes may be made therein without departing from the invention as set forth in the claims.

What is claimed is:

1. An intervertebral disc for a prosthetic intervertebral disc system for insertion from a posterior of the spine, the disc comprising:
first and second end plates sized and shaped to fit within an intervertebral space, each end plate having a vertebral contacting surface and an inner surface;
a first bearing surface on an inner surface of the first end plate;
a second bearing surface on an inner surface of the second end plate; and
a mobile core configured to be received between the first and second bearing surfaces, the first and second end plates being articulable and rotatable relative to each other via sliding motion of at least one of the first and second bearing surfaces over the core, and the core being movable with respect to at least one of the first and second end plates.

2. The intervertebral disc as set forth in claim 1, wherein the mobile core is a rigid core having at least one curved bearing surface for contacting a corresponding first or second bearing surface.

3. The intervertebral disc as set forth in claim 1, wherein the first and second end plates are configured to be arranged within a disc space to provide about ±2 to ±12 degrees of axial rotation about an axis of the core.

4. The intervertebral disc as set forth in claim 1, wherein the first and second end plates are configured to be arranged within a disc space to provide about ±5 to ±15 degrees of motion in the flexion/extension direction.

5. The intervertebral disc as set forth in claim 1, wherein the first end plate includes a projection which engages with a recess of the core to retain the core between the first and second end plates.

6. The intervertebral disc as set forth in claim 5, wherein the projection is an annular rim and the recess is an annular groove.

7. The intervertebral disc as set forth in claim 5, wherein the projection extends less than 360° around the core.

8. The intervertebral disc as set forth in claim 1, wherein the core is movable with respect to both the first end plate and the second end plate.

9. The intervertebral disc as set forth in claim 1, wherein the first and second end plates each include an elongated fin on the vertebral contacting surface thereof.

10. A prosthetic intervertebral disc system for insertion from a posterior of the spine, the disc system comprising:
first and second intervertebral discs configured to be inserted from a posterior of the spine, each of the first and second intervertebral discs comprising:
first and second end plates sized and shaped to fit within an intervertebral space, each end plate having a vertebral contacting surface and an inner surface;
a first bearing surface on an inner surface of the first end plate;
a second bearing surface on an inner surface of the second end plate; and
a mobile core configured to be received between the first and second bearing surfaces, the first and second end plates being articulable and rotatable relative to each other via sliding motion of at least one of the first and second bearing surfaces over the core, and the core being movable with respect to at least one of the first and second end plates.

11. The disc system as set forth in claim 10, wherein the mobile core is a rigid core having at least one curved bearing surface for contacting a corresponding first or second bearing surface.

12. The disc system as set forth in claim 10, wherein the first and second intervertebral discs are identical.

13. The disc system as set forth in claim 10, wherein the first and second intervertebral discs are mirror images of one another.

14. The disc system as set forth in claim 10, wherein the first and second intervertebral discs are configured to be arranged within a disc space to provide, for each of the first and second discs, about ±2 to ±12 degrees of axial rotation of the first and second end plates about an axis of the core.

15. The disc system as set forth in claim 10, wherein the first and second intervertebral discs are configured to be arranged within a disc space to provide, for each of the first and second discs, about ±5 to ±15 degrees of motion in the flexion/extension direction of the first and second end plates with respect to each other.

16. The disc system as set forth in claim 15, wherein the first end plate includes a projection which engages with a recess of the core to retain the core between the first and second end plates.

17. The disc system as set forth in claim 15, wherein the projection is an annular rim and the recess is an annular groove.

18. The disc system as set forth in claim 10, wherein the first and second end plates each include an elongated fin on the vertebral contacting surface thereof.

19. The disc system as set forth in claim 10, wherein, for each of the first and second discs, the core is movable with respect to both the first end plate and the second end plate.

20. A prosthetic intervertebral disc system for insertion between two vertebrae from a posterior of the spine, the disc system comprising:
a first intervertebral disc including a first end plate having a length no greater than a length from the anterior to the posterior of the vertebrae, a second end plate coordinating with the first end plate and a first rigid core configured to be movably seated between the first and second end plates; and
a second intervertebral disc including a third end plate having a length no greater than a length from the anterior to the posterior of the vertebrae, a fourth end plate coordinating with the third end plate and a second rigid core configured to be movably seated between the third and fourth end plates.

21. A method of inserting a prosthetic intervertebral disc from a posterior of the spine, the method comprising:
accessing an intervertebral disc space between a first and second vertebrae from a posterior of a patient;
inserting first and second intervertebral discs from the posterior of the patient on first and second sides of a midline of the spine, wherein the first and second intervertebral discs each have a mobile core.

22. The method as set forth in claim 21, comprising providing motion between the first and second vertebrae in the directions of flexion/extension, lateral bending, and axial rotation.

23. The method as set forth in claim 21, wherein the first and second intervertebral discs are inserted and placed in the intervertebral disc space without contacting one another.

24. The method as set forth in claim 21, wherein the first and second intervertebral discs are not connected to one another in the intervertebral disc space.
